(19) United States
(12) Patent Application Publication
(10) Pub. No.: US 2009/0305230 A1
(43) Pub. Date: Dec. 10, 2009

(54) Real Time Detection of Molecules, Cells and Particles Using Photonic Bandgap Structures

(76) Inventors: Kenneth L. Beattie, Crossville, TN (US); Ronald J. Tonucci, Waldorf, MD (US)

Correspondence Address: Adler & Associates, 8011 Candle Lane, Houston, TX, 77071, US

(21) Appl. No.: 12/226,036

(22) PCT Filed: Apr. 6, 2007
(86) PCT No.: PCT/US2007/008731

§ 371 Date: May 4, 2009

Related U.S. Application Data
(60) Provisional application No. 60/789,754, filed on Apr. 6, 2006.

(51) Int. Cl. C12Q 1/70; G02B 6/00; C12Q 1/68; G01N 33/53; G01N 33/567; C12Q 1/02; C12M 1/34; G01N 21/00

(52) U.S. Cl. 435/5; 385/12; 435/6; 435/7.1; 435/7.2; 435/29; 435/288.7; 356/436

(57) ABSTRACT

Provided herein is a photonic bandgap (PBG) detector effective to detect inorganic molecules, organic biomolecules or biopolymers, cells, subcellular organelles, and particles. The PBG detector utilizes photonic crystals having a binding agent attached to channel surfaces comprising the crystals to selectively bind a molecule, cell or particle of interest so that an increase in light transmission is detectably induced within the photonic bandgap upon binding. Also provided are methods of optically detectiing an analyte and of identifying the presence of a cell or a particle in a biological sample.

### BACKGROUND OF THE INVENTION

1. Field of the Invention

The present invention relates generally to the fields of molecular biology, biochemistry, genomics, proteomics, nanotechnology and analytical chemistry. More specifically, the present invention relates to a novel photonic bandgap sensor device for real time detection of inorganic, organic or biological substances.

2. Description of the Related Art

Micro- and nanoscale fabrication processes are revolutionizing the electronics and biomedical fields, enabling miniaturized analytical instrumentation for numerous industrial and medical applications. The impact of micro- and nanofabrication technology in biomedical research can be seen in the increasing presence of miniaturized analytical instrumentation in research and clinical laboratories. The emergence of “lab-on-a-chip” and similar automated small-scale instruments is expected to benefit the fields of genomics, proteomics, metabolomics, medical and environmental diagnostics, toxicology screening, food and water safety, forensics and drug discovery. Other analytical applications that would greatly benefit from microanalytical instrumentation include genetic screening, monitoring chemical and biological warfare agents, and industrial process control.

Most of these analytical applications involve detecting and monitoring a binding reaction, comprising the interaction between a support-bound “probe,” typically a specific antibody, oligonucleotide, ligand or receptor molecule, and the “target” analyte, typically a protein, nucleic acid, macromolecule, chemical species, living cell or particle. A widely used analytical procedure in genomic analysis, illustrative of such applications, is hybridization of surface-immobilized oligonucleotide probes with DNA or RNA analytes. Similarly, in proteomic and immunodiagnostic analyses, binding of surface-immobilized antibodies to proteins or other antigen-containing analytes is commonly detected.

However, current miniaturized binding assays, including both DNA and protein microarrays, suffer from several limitations which impede their widespread use and make certain applications impossible. Included are the requirement to introduce a label into the target analyte in order to enable detection and quantification, the need to analyze relatively large samples, requirement to provide target or signal amplification to enable detection of low quantities of analyte, and high cost of complex instrumentation. “Label-free” analytical instruments are available, employing surface plasmon resonance (SPR) sensors, but these are expensive and require relatively high concentrations of analyte. Detection sensitivity is a critical issue, particularly with small sample volumes. The current detection limit of DNA and protein array devices is typically hundreds to thousands of target molecules. Although this level of sensitivity is adequate for some types of samples, it is a prohibitive limiting factor for minute sample volumes (nanoliter to picoliter range), or for larger samples (milliliter to microliter range) containing very small numbers of target molecules. There is currently no device which can approach single molecule detection over a wide range of sample volumes.

The effects of optical diffraction have been studied since the time of Newton. More recently, the properties of energy vs. wavenumber or bandgap generated by highly periodic structures containing long range order have been studied extensively. In the case of photons, these structures have been called photonic bandgap (PBG) structures or photonic crystals, which mimic the band properties of their semiconductor counterparts. For PBG structures containing low loss materials, the periodic structure of the PBG is critical in determining its reflection and transmission properties. When a local variation, i.e., defect, is introduced into the periodic structure, the coherence of the photon field is locally perturbed and the reflection and transmission properties can become modified at a specific wavelength(s).

There is a recognized need in the art for improved devices for detecting and quantifying molecules, cells or particles, having improved detection sensitivity. Specifically, the prior art is deficient in simpler and more cost effective analytical devices that approach single molecule detection sensitivity over a wide range of sample volumes without labeling the analyte or amplifying the target or signal. More specifically, the prior art is deficient in the real-time simultaneous detection of a multiplicity of binding reactions using photonic bandgap structures. The present invention fulfills this longstanding need in the art.

### SUMMARY OF THE INVENTION

The present invention is directed to a a photonic bandgap (PBG) detector. The PBG detector comprises one or more photonic crystals having a matrix structure defining a plurality of channels having a length I therethrough, a fluid within the channel(s) and flowable therethrough and means for biochemically inducing a detectable increase in light transmission within a bandgap region of the photonic crystal. The PBG detector also comprises means for transmitting light within the bandgap region and means for detecting the increase in light transmission therein. A related PBG detector further comprises an optical filter operably disposed between the photonic crystal and the means for detecting the increase in light transmission. Another related PBG detector further comprises means for applying a variable back pressure to the flow through the channels.

The present invention also is directed to a method of optically detecting an analyte. The method comprises flowing an analyte through the channels of the photonic crystal described supra, illuminating the photonic crystal with a light source and inducing an increase in light transmission within a photonic bandgap of the photonic crystal via binding of one of the analytes to a first binding agent attached to surfaces of the channels and specific for the analyte. The increase in light transmission within the photonic bandgap is photodetected thereby optically detecting the analyte. A related optical detection method further comprises flowing the analyte through the channels of a series of photonic crystals each comprising a different first binding agent effective to bind the one analyte. Another related optical detection method further comprises flowing a reporter having a second binding agent effective to bind the analyte attached thereto through the channels, where the first and second binding agents are effective to bind the analyte concurrently without binding to each other. Yet another related optical detection method further comprises applying a variable back pressure to the flow through the channels thereby decreasing non-specific analyte binding.

The present invention is directed further to a method of identifying the presence of a cell or particle in a biological sample. The method comprises flowing the biological sample through the channels of one or more photonic crystals described supra where the channel surfaces comprise one or more antibodies specific for the cell or particle attached thereto, illuminating the one or more photonic crystals with a light source and photodetecting an increase in light transmission within a photonic bandgap of the photonic crystal(s) upon binding of the cell or particle to the one or more antibodies thereby identifying the cell or particle. In a related method the cell is a lymphocyte from an individual where the method comprises determining the types of antibodies to which the lymphocyte binds to indicate exposure of the individual to a pathogen or to indicate a change in immunocompetent status of the individual.

Other and further aspects, benefits and advantages of the present invention will be apparent from the following description of the presently preferred embodiments given for the purpose of disclosure.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1D illustrates the operation of (FIGS. 1A-1C) and various geometries of (FIG. 1D) a PBG detector.

FIGS. 2A-2B illustrate the PBG device function (FIG. 2A) and multi-detection scheme (FIG. 2B).

FIG. 3 illustrates a side illumination and vertical detection method and waveguiding.

FIGS. 4A-4B illustrate the use of long duplex DNA spacer molecules to enable movement of bound cells or particles to facilitate multiple bindings to the channel sidewalls.

FIG. 5 illustrates a method for hybridization-directed arraying of antibodies onto separate photonic crystal elements of the PBG sensor.

FIGS. 6A-6B depict the flow of particles through a photonic crystal containing long channels and index contrast caused by capture of a target cell. FIG. 6A illustrates the flow of particles plus liquid through photonic crystal channels. FIG. 6B illustrates a target captured in the top of the channel (blue) blocking the flow of particles through the channel.

### DETAILED DESCRIPTION OF THE INVENTION

In one embodiment of the present invention there is provided a photonic bandgap (PBG) detector, comprising one or more photonic crystals having a matrix structure defining a plurality of channels having a length I therethrough; a fluid within the channel(s) and flowable therethrough; means for biochemically inducing a detectable increase in light transmission within a bandgap region of the photonic crystal; means for transmitting light within the bandgap region; and means for detecting the increase in light transmission therein.

Further to this embodiment the PBG detector may comprise an optical filter operably disposed between the photonic crystal and the means for detecting the increase in light transmission. In another further embodiment the PBG detector may comprise means for applying a variable back pressure to the flow through the channels. In all these embodiments the said photonic crystals may be arranged in series.

Also in these embodiments the means for biochemically inducing a detectable increase in light transmission may comprise a molecule, a cell or a particle flowably disposed within the channels. Examples of a molecule are an inorganic molecule, DNA, RNA, a protein, a toxin, a prion, a peptide, a receptor, or other biomolecule. Examples of a cell are a bacterium, archaea, an amoeba, a protist, mycoplasma, a microfungus, a microalgae, a microparasite, a virus, a lymphocyte, or a subcellular organelle. Examples of a particle are a spore or a virus.

Furthermore, the PBG detector comprises a first binding agent attached to surfaces of the channels effective to bind molecule, cell or particle thereto. The first binding agent may be DNA, an antibody, a peptide, or a DNA conjugate thereof. In one aspect the first binding agent may a DNA-antibody conjugate. Further to this aspect the DNA comprising the DNA antibody conjugate may be hybridized to a DNA attached to the channel surfaces. Further still, the PBG detector may comprise a second binding agent attached to a reporter where the second binding agent is incapable of binding to the first binding agent and effective to bind the molecule, cell or particle at a site separate from the first binding agent. An example of a reporter is a nanoparticular or microparticular structure. The second binding agent may be a DNA, an antibody or a peptide.

In an aspect of this embodiment the PBG detector may comprise means for enhancing the detectable increase in transmitted light. In this particular aspect the means for enhancing the detectable increase in transmitted light may comprise the plurality of channels having a length greater than the length I where the fluid therein includes a plurality of flowable nanoparticles having a diameter less than a wavelength of the transmitted light and the first binding agent is attached to the surfaces of the channels at a position substantially proximate to a top end thereof. The nanoparticles may have a diameter about 10 to about 100 times less than a wavelength of the transmitted light.

In all aspects of these embodiments the fluid may be a gas or a liquid. An example of a gas is moist air, helium, hydrogen, nitrogen, argon, krypton, or sulphurhexafluoride. Representative examples of a liquid are water, a buffer, an ionic solution, a sugar solution or a suspension of nanoparticles having a diameter about 10 to about 100 times less than the wavelength of transmitted light.

In another embodiment of the present invention there is provided a method of optically detecting an analyte, comprising flowing an analyte through the channels of the photonic crystal described supra; illuminating the photonic crystal with a light source; inducing an increase in light transmission within a photonic bandgap of the photonic crystal via binding of one of the analytes to a first binding agent attached to surfaces of the channels and specific for the analyte; and photodetecting the increase in light transmission within the photonic bandgap thereby optically detecting the analyte.

Further to this embodiment the method may comprise applying a variable back pressure to the flow through the channels thereby decreasing non-specific analyte binding. In another further embodiment the method may comprise flowing the analyte through the channels of a series of photonic crystals each comprising a different first binding agent effective to bind the one analyte. In yet another further embodiment the method may comprise flowing a reporter having a second binding agent effective to bind the analyte attached thereto through the channels, where the first and the second binding agents effective to bind the analyte concurrently without binding to each other.

In all of these embodiments the analyte may be those molecules and cells as described supra. Also, in all embodiments the reporter and the first and second binding agents are as described supra.

In yet another embodiment of the present invention there is provided a method of identifying the presence of a cell or particle in a biological sample, comprising flowing the biological sample through the channels of one or more photonic crystals described supra, the channel surfaces comprising one or more antibodies specific for the cell or particle attached thereto; illuminating said one or more photonic crystals with a light source; and photodetecting an increase in light transmission within a photonic bandgap of the photonic crystal(s) upon binding of the cell to the one or more antibodies, thereby identifying the cell or particle.

In this embodiment the antibody may be conjugated to a DNA attached to the channel surfaces. Furthermore, the DNA comprising the DNA antibody conjugate is hybridized to another DNA attached to the channel surfaces.

Also in this embodiment the cell or particle may be a bacterium, archaea, an amoeba, a protist, mycoplasma, a spore, a yeast, a microfungus, a microalgae, a microparasite, a virus, or a lymphocyte. In one aspect the cell or particle may be a pathogen. Examples of a pathogen are E. coli O157:H7 or M. tuberculosis, Bacillus anthracis, Francisella tularensis, Yersinia pestis, Yersinia enterocolitica, Campylobacter jejuni, Listeria monocytogenes, Salmonella species, Shigella species, Vibrio species, Burkholderia mallei, Burkholderia pseudomallei, Coxiella bumetii, Brucella species, Chlamydia species, Coccidioides posadasii, Rickettsia proawzekii, Rickettsia rickettsii, smallpox virus, hemorrhagic fever viruses, encephalitis viruses, yellow fever virus, rabies virus, severe acute respiratory syndrome-associated coronavirus (SARS-CoV), Chikungunya virus, bird flu (H5N1 influenza) virus, Noroviruses, hepatitis viruses, calciviruses, West Nile virus, encephalitis viruseses, prions, Cryptosporidium parvum, Cyclospora cayatanensis, Giardia lambia, Entamoeba histolytica, Toxoplama, and Microsporidia. In another aspect of this embodiment the cell may be a lymphocyte from an individual where the method further comprises determining the types of antibodies to which the lymphocyte binds to indicate exposure of the individual to a pathogen or to indicate a change in immunocompetent status of the individual.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

Provided herein is a real time detection device and methods of use in which a bacterium, other cell, or particle, when introduced into the periodic structure of a photonic bandgap (PBG) structure, modifies the coherence of the photon field locally, creating a detectable increase in transmission within the “forbidden” or highly reflective bandgap region of the PBG structure. Since a bacterium, other cell, or particle placed within the PBG structure itself can provide the local variation in the coherence of the periodic structure, the modification in the transmission properties due to this local variation is considered real time. That is, the object of interest actually becomes an integral part of the detection mechanism, either transiently, as the material is flowed through the PBG structure, or constantly, if the interior surface of the photonic crystal is coated with a binding reagent specific to a given object of interest, such as an antibody or nucleic acid probe.

Particularly, the present invention provides a photonic crystal sensor device and methods for its use in detecting molecules, cells and particles. The device comprises a photonic crystal with throughput channels of a length I extending between the upper and lower surfaces thereof. The channels, which may be filled with gas or liquid, have a different index of refraction from the surrounding matrix material. A suitable gas may be moist air, a noble gas, such as helium, hydrogen, nitrogen, argon, krypton, or a heavy dense gas, such as sulphurhexafluoride. A suitable liquid may be water, a buffer, an ionic solution, a sugar solution or a suspension containing nanoparticles having a diameter about 10 times to about 100 times smaller than the wavelength of the incident light.

The photonic crystal exhibits a photonic band gap such that transmission of light of a defined spectral bandwidth at specific incident angles through the crystal is forbidden. Specific binding agents or reagents are immobilized to the sidewalls of the channels within discrete photonic crystal elements. Photonic crystal elements may be laterally located in linear or two-dimensional arrays.

Molecules, cells or particles passing through the channels interact with and bind to the binding reagents, resulting in a defect in the photonic crystalline structure. Thus, the index of refraction within one or more throughput channels is altered, resulting in perturbation of the photonic band gap such that light of a theretofore prohibited wavelength is transmitted through the crystal and detected by a simple optical system such as a diode detector. Binding of the sample component to the binding agent in different photonic crystal elements is detected, providing information about sample composition. The geometry of the throughput channels can be tuned to enable the photonic crystal sensor to detect the binding of cells, spores, or inert nanoparticles of a wide range of sizes.

For detection of molecular species, a sandwich binding assay is provided, wherein a first binding agent is immobilized on the sidewalls of throughput channels, a second binding reagent is immobilized on the surface of a “reporter”, e.g. a micro- or nanoparticle of dimensions slightly smaller than that of the throughput channel. The analyte molecule binds to both first and second binding reagents to capture the particle within the photonic crystal, thereby perturbing the photonic band gap. The device provides the ability to detect the binding of a single, or small number of analytes, such as, molecules, cells or particles within each photonic crystal element of the sensor.

An important feature of the PBG sensor is that it can directly detect the presence of a cell or particle without the need to introduce a label into the analyte. Modifications to this scheme, involving the use of “reporter particles” coated with binding agents, allow for the real time detection of much smaller objects, for example, viruses, biopolymers, such as nucleic acids and proteins, and yet smaller organic and inorganic molecules. It is contemplated that even single cells, particles and molecules can be detected by the PBG structure device.

An important functional feature of the PBG sensor device is the ability to apply a positive or negative back pressure to the PBG while under biological or biochemical interrogation. This back pressure allows for a significant reduction in non-specific binding, a cause of false positive responses in many biological and chemical detections systems. By applying controlled variations to the back pressure of the device, variations in environmental states such as temperature or ionic conditions, or combinations of these variations, the PBG structure can also be used for analytical studies of the binding energies of the above mentioned entities. Such application of physical and/or chemical forces also allows for the release of captured entities for further analysis or resetting of the device for additional detection cycles. It is contemplated that the PBG sensor device may be operated under either liquid or gaseous flow conditions.

Photonic crystals may be made from a variety of transparent or nearly transparent materials near the resonant frequency of the device. They include, but are not limited to, glass, plastic, polymer, semiconductor, insulator, conducting, such as the family of indium tin oxide ITO compounds, naturally occurring minerals, such as mica or quartz, non-linear optical materials, meta-materials, and amorphous materials. For materials with a high index of refraction, the minimum distance light is required to traverse the photonic crystal and still be able to set up a coherence field acceptable for defect signal detection may be as short as a 5-7 array spacings. Therefore, materials containing a high absorption coefficient may still be suitable for device fabrication providing they possess an appropriately high index of refraction and contain channels composed of appropriate low index of refraction materials, e.g., air or water.

It is contemplated that using hydrophilic polymers, particularly polymers comprising a multiple component system, such as, but not limited to, 4 components, may reduce the cost of PBG structures to almost zero. These hydrophilic polymers allow the passage of water for detection of analytes in aqueous solutions. It also is contemplated that the commercially available multi-functional epoxy-based polymer SU-8, a glycidyl ether derivative of bisphenol-A novolac, is highly suitable for use as a low cost, easily manufactured photonic crystal.

The geometry of the photonic crystal will affect the band structure and polarization of the transmission, i.e., reflection, spectra and hence the resonant wavelength of the device. In a 2 dimensional device the geometric structure may be hexagonal close packed (HCP or triangle lattice), square or other highly symmetric geometry for ease of signal determination and characterization. Highly symmetric geometries are well known and standard in the art.

Preferably, the size of a channel comprising an element of the array in a 2 dimensional system should be on the order of the size of the target, which may be, but not limited to, a particle, cell, bacterium, sphere, spore, or virus, used to produce the defect. As the target size becomes small compared to the volume of the channel or its index of refraction approaches that of the channel, the amount of transmitted or reflected signal light will be reduced. A high density of channels with aspect ratio, i.e., the ratio of channel length to channel diameter, approaching that of the target is preferred for best signal efficiency.

Photonic crystal band structure can be detected easily with an index of refraction difference between the matrix and channel material as small as 0.002. The index of refraction of the target comprising the defect preferably is different from that of the channel material. The index of refraction, channel diameter and array spacing play a critical role in determining the band structure and location of the defect transmission and reflection spectrum. The higher the index contrast between the matrix and channel material, the more rapid the required coherence length of the photon is established. Hence, a low index contrast between the matrix and channel material is preferred for a high throughput system containing many channels while a higher index contrast is preferred when a strong signal is required.

The geometry of this device may also be extended to 1 dimensional, as in a series of plates, or 3 dimensional structures, such as cubic, e.g., simple, BCC or FCC, HCP or tetragonal, by anyone skilled in the art of producing similar photonic crystal structures and bandgaps for which a target passing through the system would perturb the coherence of the photon field resulting in a “defect site” thereby allowing passage of a detectable signal in transmission or reflection as described herein.

Thus, the present invention provides methods for detecting the presence of and further identifying analytes, such as, molecules, cells or particles. Specific molecules may include an inorganic molecule, DNA, RNA, a protein, a toxin, a prion, a peptide, a receptor, or other biomolecule. Cells may include a bacterium, archaea, an amoeba, a protist, mycoplasma, a yeast, a microfungus, a microalgae, a microparasite, or a lymphocyte. Particles not considered in the art to be cells or to have a cellular structure may include a spore or a virus.

Also, the photonic bandgap structures provided herein are effective to detect pathogens. Particularly, the PBG structures are use for detecting CDC Select Biothreat Agents and NIAID Category A, B & C Priority Pathogens. For example, without being limiting, the PBG structures may detect the presence of E. coli O157:H7 or M. tuberculosis, Bacillus anthracis, Francisella tularensis, Yersinia pestis, Yersinia enterocolitica, Campylobacter jejuni, Listeria monocytogenes, Salmonella species, Shigella species, Vibrio species, Burkholderia mallei, Burkholderia pseudomallei, Coxiella bumetii, Brucella species, Chlamydia species, Coccidioides posadasii, Rickettsia proawzekii, Rickettsia rickettsii, smallpox virus, hemorrhagic fever viruses, encephalitis viruses, yellow fever virus, rabies virus, severe acute respiratory syndrome-associated coronavirus (SARS-CoV), Chikungunya virus, bird flu (H5N1 influenza) virus, Noroviruses, hepatitis viruses, calciviruses, West Nile virus, encephalitis viruseses, prions, Cryptosporidium parvum, Cyclospora cayatanensis, Giardia lambia, Entamoeba histolytica, Toxoplama, and Microsporidia.

The following examples are given for the purpose of illustrating various embodiments of the invention and are not meant to limit the present invention in any fashion.

### EXAMPLE 1

### Real Time Detection of Cells Using Photonic Bandgap Structures

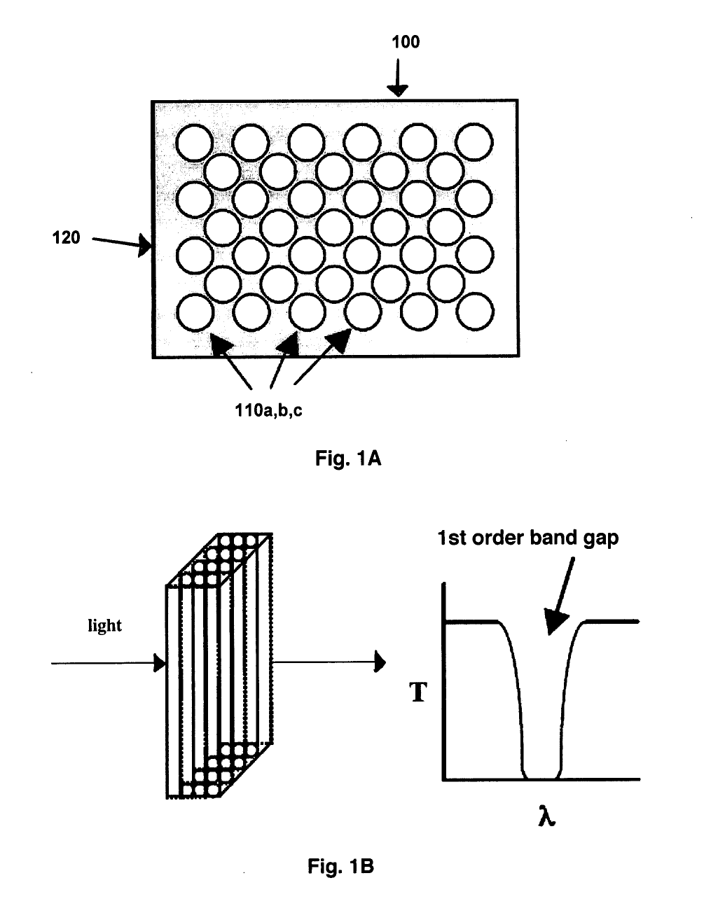

The observation of photonic crystal defects optically depends upon the orientation of the photonic crystal and the direction of observation. For the devices described in these Examples, detection of defect structures is either along an axis of high symmetry, exhibiting the simplest band structure or vertically, perpendicular to a high symmetry axis for high throughput devices. The operation of a 2-dimensional PBG structure to detect a cell, such as a bacterium or lymphocyte is shown in FIGS. 1A-1C. FIG. 1D shows various possible geometries, i.e., 1-, 2- and 3-dimensional, of PBG structures.

FIG. 1A shows a 2-dimensional hexagonal close packed structure 100 containing uniform holes 110a,b,c or channels in a dielectric matrix 120. The dielectric medium is considered transparent in the region of interest. The holes represent approximately 40 percent open area for the region containing them. Such a structure would exhibit a photonic band gap for an index of refraction (n) difference, between the channels and the matrix material, of 0.02 to 3 and beyond as shown in the transmission spectra of FIG. 1B.

In this example, light is directed along a high symmetry mirror plane of the periodic structure and the light not transmitted in the band gap region is reflected from the front surface of the PBG structure. Additional, higher order band gaps would appear at shorter wavelengths (I). If a movable rod were placed into a channel filling its volume, the coherence of light passing near this area would be modified locally, allowing for some of the light to pass in the spectral region of the band gap, instead of being reflected. The amount of light transmitted within the spectral region of the band gap would depend on the index of refraction of the rod compared with that of the unoccupied channel.

If a rod were inserted into a channel whose length was shorter than the length of the channel, or if the rod filled less than the entire length of the channel, it is anticipated that the amount of light transmitted within the band gap region would be attenuated. If the rod were replaced by another object whose dimensions equaled or nearly equaled the volume of the channel, a similar modification to the amount of light passing through the band gap region would occur. The list of materials that could cause leakage of light in the bandgap region is extensive, including, but not limited to, inorganic, organic, biological or other materials whose index of refraction is different from that of the material filling the channels, which in this example, is air.

For the case of a biological structure replacing the rod, such as a single cell or bacterium, a modification to the light transmitted within the band gap region would occur as described above, allowing detection of a single cell during its occupation within the PBG structure. This additional light could be captured by a photo detector providing a signal that the cell is within the PBG structure. Each time a cell enters and leaves the PBG structure, a similar signal would be generated, providing a method to count cells as they pass through the structure.

To detect the presence of a specific type of cell or bacterium occupying the PBG structure, the sidewalls of the channels within the matrix material may be coated with a binding reagent, eg., antibody or other protein, such that only a specific type of cell would react with the substance causing the cell to bind strongly to the sidewalls of the channel. Instead of the cell passing through the PBG structure, it would remain within the structure, producing a constant signal to the photo detector indicating a binding reaction had taken place, thus identifying the type of cell captured (FIG. 1C).

A typical bacterium is a rod of diameter about 0.2 to about 1 micron in diameter and about 1 to about 5 microns in length, although bacteria of diameter as small as 100 nanometers and length as large as 20 microns or greater are known. Cell length increases as they grow and approach cell division, so the effective length varies over a two-fold range. Some species of bacteria tend to form pairs, tetrads or chains, which alters their effective dimensions, in which case photonic crystal channel diameters and flow conditions need to be carefully adjusted to accommodate these situations.

In addition, some bacteria have pili or flagella attached to their surfaces, which may increase their effective dimensions and influence their ability to readily pass through the photonic crystal channels, but also may present useful antigens for capture by specific antibodies coating the channels. Furthermore, some bacteria produce slimy capsid coatings, containing mucopolysaccharides and other substances which may increase their effective size and/or cause nonspecific binding to the channel sidewalls. In these instances the bacteria may be pre-treated with chemical and physical means to remove these capsid materials to yield a more uniform cell size and reduce nonspecific binding.

A bacterium of particular interest to detect using the PBG sensor device is Mycobacterium tuberculosis, which is a rod of about 0.3-0.4 microns in diameter and about 2 microns in length. For detection of this bacterium a photonic crystal containing channels of about 500 nm diameter would be appropriate. Monoclonal antibodies specific to cell-surface antigens on Mycobacterium tuberculosis are available commercially which may be attached to the surface of the photonic crystal channels using a variety of well known methods.

Another bacterial species that is of particular clinical importance is Escherichia coli O157:H7, which causes life-threatening hemorrhagic colitis and is widespread in the environment and in contaminated meats. E. coli is about 0.75 microns in diameter and about 2 microns in length. Monoclonal antibodies specific to the clinically important O157:H7 strain are available for attachment to the sidewalls of the PBG channels. For detection of E. coli O157:H7 a photonic crystal containing channels of about 800 nm diameter would suffice.

Eukaryotic cells vary widely in shape and size, depending on species and tissue type, but the typical diameter is about 10-20 microns. A cell of particular interest with regard to biological significance which is detectable using the PBG sensor device is the lymphocyte, which is typically 8-10 microns in diameter and displays on its cell surface antibody-like receptors. In a blood sample the spectrum of receptors and other proteins on the surface of “immune cells” reflects the history of the individual's exposure to pathogens, as well as the status of the immune system of he individual.

Lymphocytes collected from an individual also may be exposed to various chemical or biological agents in vitro and the changes in cell surface receptors and other proteins monitored to further assess immunological status or study the effect of immunomodulatory therapies. Therefore, it would be particularly useful to monitor the spectrum of immune cell surface proteins and receptors, using an array of photonic crystal elements in which the channels are coated with peptide or proteins that are recognized and bound by cell surface receptors, or coated with specific antibodies that recognize and bind to cell surface proteins of interest.

Sufficient additional pressure or other physical or chemical means could be applied to break the bond between the cell and the reagent applied to the side wall of the channels to allow the cell to leave the PBG structure. The cell could then be captured externally from the PBG structure for further analysis. For analysis of eukaryotic cells, the preferred medium containing the cells to be passed through the photonic crystal device would comprise an aqueous buffer, which could be modified by the addition of substances such as sugars or soluble polymers to modify its index of refraction from that of the cell and still remain environmentally friendly to the cell. Alternatively, the cells could be aerosolized using a nebulizer or atomizer before introduction into the PBG device and the binding reagents on the sidewalls of the channels could be kept moist while the cells pass through the PBG structure.

Other types of biological objects that may be detected by this method, using photonic crystals having channels slightly larger than the objects of interest, include, but are not limited to, archaea, amoeba, protozoa and other protista, mycoplasma, spores, yeasts, microfungi, microalgae, microparasites, viruses, and subcellular organelles.

### EXAMPLE 2

### Real Time Detection of Spherical or Cyllindrical Particles Using Photonic Bandgap Structures

If the rod in Example 1 were replaced by a sphere of diameter slightly smaller than that of the channels of the PBG structure, a similar transmission of light is induced within the band gap while the sphere resides within the PBG structure. For this example it is assumed that the length of the channel is also on the same order as the diameter of the channel. The side walls of the PBG channels and spheres may be chemically modified to provide a thin coating of specific binding reagents. The specific binding reaction causes a particular surface modified sphere to stick to the side wall of the channel as it passes through the PBG structure preventing the sphere from moving out of the channel. As in Example 1, a photo detector may be utilized to detect capture of a sphere within the PBG structure indicating a binding reaction has taken place. Spheres lacking the specific chemical modification to their surfaces pass through the PBG structure resulting in pulses of light at the photo detector, producing a sphere counter. Similarly, cylindrical materials, i.e., short rod-shaped particles, of diameter slightly smaller than that of the channels of the PBG structure will behave similarly to the spheres.

### EXAMPLE 3

Use of “Reporter Particles” to Detect Smaller Objects or Substances

The binding reaction between the surface of the channel and a spherical or cylindrical particle in Example 2 may be modified to provide a particularly useful embodiment of the PBG sensor device, in which a “reporter particle” is provided which functions to detect much smaller substances. The side walls of the PBG channels are chemically modified with a thin reactive coating of type A and the surface of the spherical or cylindrical “reporter particle” is chemically modified by a thin reactive coating of type B. The coatings A and B are selected to be nonreactive, i.e., do not bind, to each other, but so bind specifically to separate sites on a third molecule, cell, particle or other substance of interest, e.g., the analyte, whose dimensions are small compared to the channel and reporter particle diameters.

If a reporter particle coated with reagent B is introduced into the PBG structure, it will pass through the structure unencumbered. However, if the reporter particle is introduced in the presence of the analyte particle or substance capable of binding to both A and B, then the reporter particle is captured within the PBG structure by a “sandwich” binding reaction in which the analyte particle or substance is sandwiched between the reporter particle and the side wall of the PBG channel. Capture of the reporter particle, resulting in the transmission of additional light within the band gap, would indicate the presence of a specific particle or substance, e.g., the analyte, capable of binding to both A on the sidewall and to B on the sphere.

Binding agents A and B suitable for use in this type of assay include proteins, antibodies, receptors, peptides, ligands, DNA, RNA, and any other molecule capbable of specifically binding to the analyte of interest. Analyte particles or substances that may be detected in this type of sandwich binding reaction include bacteria, cells, viruses, spores, DNA, RNA, proteins, toxins, prions, antibodies, receptors, ligands, and any other molecules or clusters containing a few atoms or less, for which specific binding reagents A and B are available. A significant advantage of PBG sensor-based detection is that the analyte does not need to be labeled with a flurorescent, chemiluminescent, radioactive, or other type of tag. A variety of materials may serve as reporter particles, including monodisperse structures, such as spheres or cylindrical rod-shaped particles of nanometer or micrometer diameter composed of latex, polystyrene, other polymers or plastics, silica, glass, metal oxides, ceramics, segments of carbon nanotubes, and vesicles, to name a few.

For example, the analyte may be a strand of DNA. A first strand of DNA complementary to a first region of the DNA analyte is tethered to the surface of the PBG channel and a second strand of DNA complementary to a second region of the DNA analyte is attached to the reporter particle. The DNA analyte is mixed with the reporter particle prior to entry into the PBG structure. Under ionic and temperature conditions favorable to DNA hybridization and known and standard in the art, the first region of the DNA analyte may bind to its complement on the surface of the reporter particle and as the complex passes through the PBG structure the second region of the DNA analyte may bind to the side wall of the channel, resulting in capture of the reporter particle within the channel, inducing transmittance of light through the PBG structure. Alternatively, the sample may be recirculated through the photonic bandgap material to allow the sample to pass multiple times through the sensor device to maximize the binding efficiency.

In the detection of DNA or RNA sequences using this “sandwich binding” assay, the preferred medium is an aqueous buffer of relatively high ionic strength, such as “standard saline citrate” (SSC) or other hybridization buffer well known and standard in the art of nucleic acid hybridization. The temperature of the reaction that is required for sequence-specific hybridization will depend on the nucleotide composition and length of the DNA probes tethered to a reporter particle and to a channel sidewall, as well known to one of ordinary skill in the art. Additional buffer components may be added to yield a refractive index that is appropriate for optical detection with the PBG structure. The flow of sample through the PBG material may be reciprocated to allow the sample to pass multiple times through the PBG sensor device to maximize the binding efficiency.

In addition, by precisely controlling of the pressure/flow rate of the medium carrying the reporter particles and DNA strands through the photonic crystal channels, the specificity of hybridization can be enhanced. Furthermore, the PBG sensor device may be used advantageously to derive kinetic and thermodynamic parameters of strand-strand, i.e., duplex, interactions, by performing the binding reaction as a function of temperature and analyte concentration and, furthermore, by quantitating the release of “reporter particles” from the PBG sensor device, as a function of temperature and/or pressure/flow rate.

In yet another example of the reporter particle application, the PBG channel and reporter particles may be coated with two different antibodies, recognizing separate antigenic determinants on a protein analyte. In this case, if a protein-containing sample is mixed with the reporter particles, then passed through the PBG material, the reporter particles are bound within the PBG structure only if the sample contains the protein analyte of interest. This assay is performed preferably in an aqueous buffer medium. Alternatively, the protein-reporter particle complexes may be formed first, then thoroughly washed to remove unbound protein. Then the mixture may be treated using a nebulizer or atomizer to form an aerosol, which is then introduced into the PBG structure under moist air conditions that allow specific antibody-antigen binding.

### EXAMPLE 4

### PBG Sensor Arrays with Photonic Crystal Elements Arranged in Series

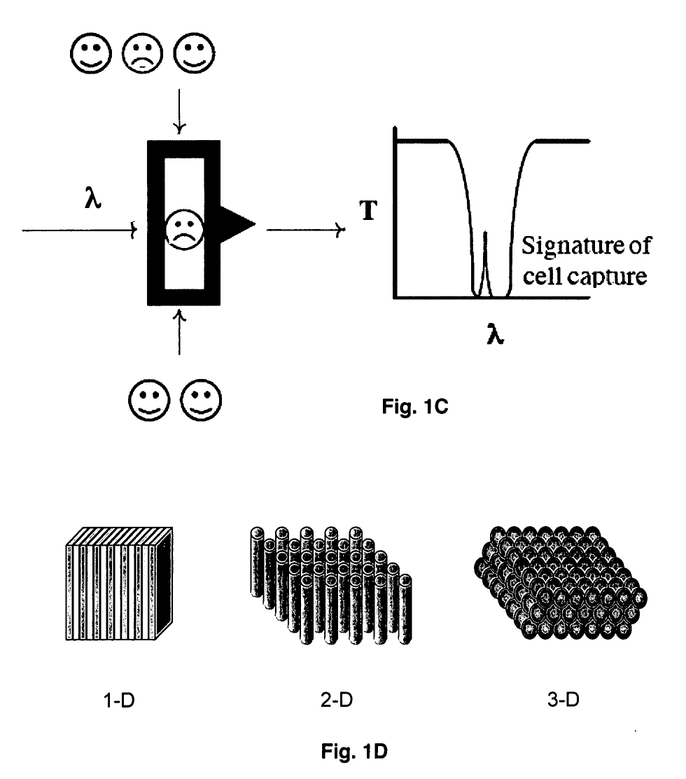

As illustrated in FIGS. 2A-2B, the PBG sensor device may be configured to enable a multiplicity of binding reactions to be monitored, whereby the analyte or analyte plus reporter particles are passed through a multiplicity of photonic crystal elements, each of which has a different binding reagent coating the sidewall channels, for example a channel of the PBG structure. In FIG. 2A these photonic crystal elements are arranged in a one-dimensional (linear) array. As shown in FIG. 2B, the detector can be further modified to provide a two-dimensional array of photonic crystals arranged in series, whereby rows of photonic crystal elements are laterally positioned and connected in a serpentine fashion. Thus, the analyte (or analyte plus reporter particles) may be passed through one row of detector elements, make a 180 deg. bend, pass through another row of detector elements, make another 180 deg. bend, etc. In this way, a single sample may be interrogated by a large number of different binding reagents, to simultaneously probe for numerous distinct features within the sample, such as different nucleic acid sequences, different proteins of interest, different cells, viruses, etc. The flow of sample through the two-dimensional device may be reciprocally controlled to maximize the binding efficiency. In addition, if a heterogeneous mixture of cell types and sizes is being analyzed, it could be advantageous to provide a pre-filter at the front end of the device, to remove cells above a certain diameter to prevent them from clogging the channels within the PBG material.

Also in this embodiment is the addition of a notch filter placed between the photonic crystal and detector. When no reporter is present in the photonic crystal, the detector sees a minimal signal as a result of the photonic band gap, assuming the spectral sensitivity of the detector only resides within the bandgap region or the source of light is only within the spectral region of the bandgap. As illustrated in FIG. 2A a notch filter is chosen such that its transmission spectrum is the inverse of the photonic band gap transmission spectrum everywhere. Hence, the total transmission of the photonic bandgap plus notch filter in series will be zero everywhere. Only when a reporter or other target object is inserted into the photonic crystal, creating a local perturbation and thus allowing light within the bandgap spectral region to pass, will the detector receive a signal. This modification can significantly reduce the cost of components for the total detector assembly allowing the light source to be as simple as a light bulb or inexpensive light emitting diode and the detector element an inexpensive diode detector.

### EXAMPLE 5

### Vertical Detection Method and Waveguiding

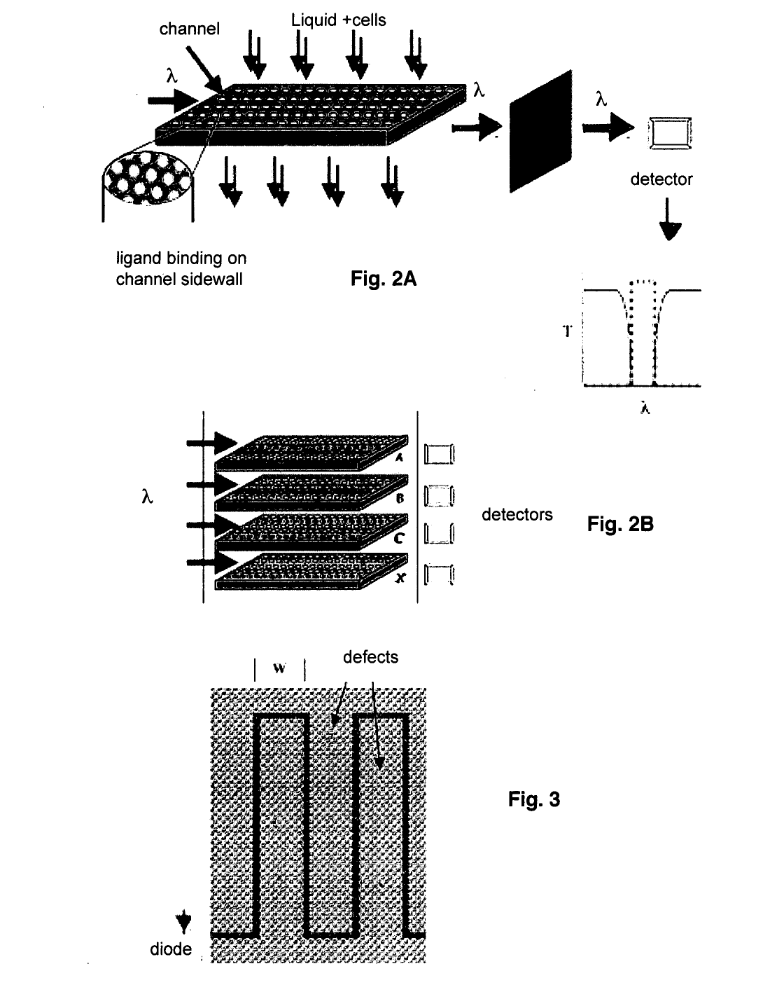

The signal from a local perturbation contained within the photonic crystal may be detected vertically by using the wave guiding properties of photonic band gap structures and off axis optical emission. A few rows of channels may be excluded from a photonic crystal structure creating a wave guide at the band gap wavelength, as illustrated in FIG. 3. The waveguide (W) within the PBG structure can be adjusted such that the local perturbations or defects described in the above examples create an evanescent wave with sufficient transverse momentum to be observed perpendicular to the plane containing the PBG structure. The average distance between waveguides must be less than twice the distance associated with the coherence disruption caused by the local perturbation. Because PBG waveguides exhibit very low loss for light whose wavelength is within the bandgap, very long waveguides are possible and hence large areas of a photonic bandgap structure can be sampled for target molecules, cells or particles by this method. The advantage of this device configuration is the ability to flow greater amounts of gases or liquids through a large area greatly increasing the total volume of sample to be characterized per unit time.

### EXAMPLE 6

### Immobilization of Binding Reagents on Throughput Channels of Photonic Crystal Elements

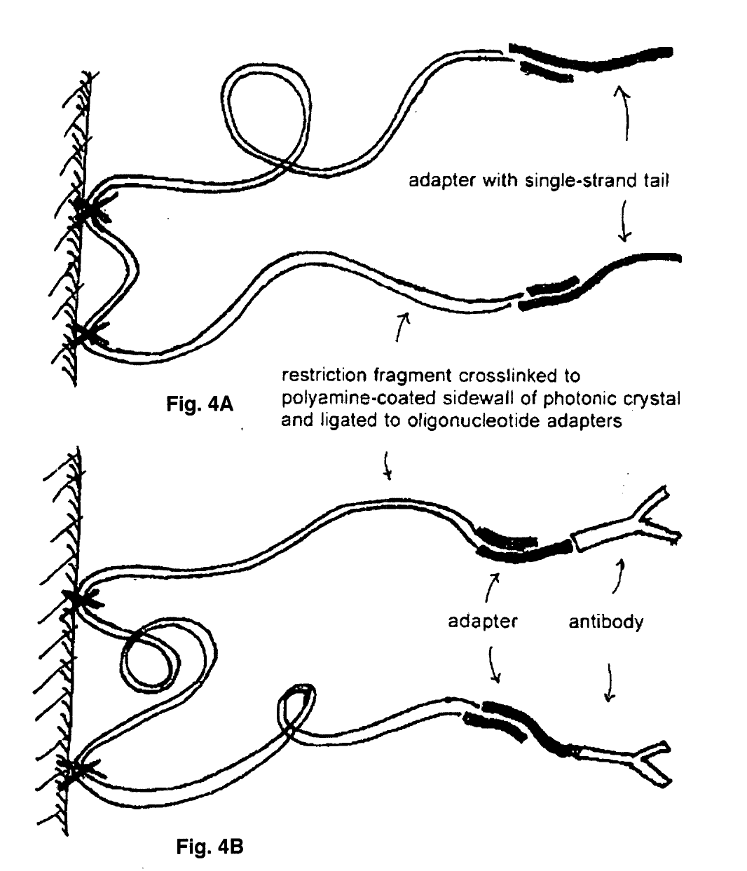

As illustrated in FIG. 4A, attachment of long, e.g., several thousand bp, DNA probes to the sidewalls of PBG channels can be advanteously used to provide “wiggle room” for reporter particles bound to the long DNA probes in the presence of analyte nucleic acid, facilitating their movement within the channels to enable multiple binding reactions per particle. For example, restriction fragments several thousand bp in length may be ligated with adapter oligonucleotide pairs to provide oligonucleotide tails at the ends. These adapter oligonucleotides may be attached to the sidwalls of the photonic crystal channels, which are coated with a polyamine film, via UV crosslinking, as is known and standard in the art. The adapter oligonucleotides may be designed to bind to reporter particles via the sandwich hybridization reaction described in Example 3, which occurs only in the presence of the analyte nucleic acid of interest. It is contemplated that other known and standard methods of attaching oligonucleotides to a surface may be useful in the present invention.

For attachment of proteins, including, but not limited to, antibodies, receptors or peptides, to derivatized surfaces, numerous methods are available. An organic film, such as reactive silane, dextran-based hydrogel, agarose, or porous polyacrylamide hydrogel, is formed on the sidewall surfaces of the PBG channels whereupon the protein is crosslinked to the film, typically via primary amines of the protein as is known and standard in the art. However, a problem with protein attachment to surfaces is that different types of proteins require somewhat different attachment conditions to avoid structural changes that may affect their ability to bind to the analyte of interest. Alternatively, for covalent attachment of antibodies to surfaces, the following approach may be used. The surface is derivatized first with thiol groups using a mercaptopropyltrimethoxysilane reagent, then activated using a heterobifunctional crosslinking reagent such as N-(γ-maleimidobutyryloxy)succinimide ester, then the antibody solution is applied to achieve covalent crosslinking to the surface.

In certain embodiments of the PBG sensor it is advantageous to immobilize DNA-antibody conjugates to surfaces, using methods for binding either the nucleic acid component or the protein component, as described above. To form the DNA-antibody conjugates, the antibody is first activated by reaction with N-(γ-maleimidobutyryloxy)sulfosuccinimide ester, then conjugated with 5′-thiol-derivatized oligonucleotide as described by the supplier (Pierce).

In one application of DNA-antibody conjugates, illustrated in FIG. 4B, long restriction fragments ligated to adapter oligonucleotides to create single-stranded tails, as described, are first crosslinked to polyamines on the surfaces of the photonic crystal channels, then hybridized with oligonucleotide-antibody complexes, and optionally ligated to form a covalent linkage, to provide “wiggle room” for enabling their movement within the channels of cells bound via antibody-antigen reacton, and facilitating multiple binding reactions per cell.

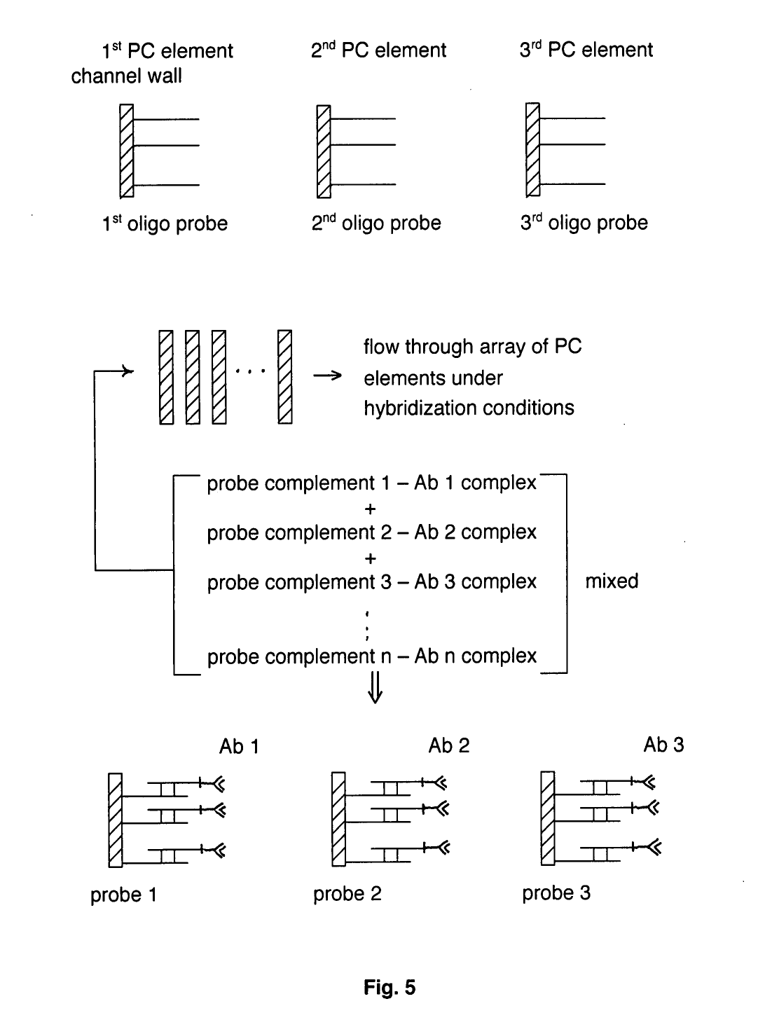

In a second application of DNA-antibody conjugates, formation of photonic crystal-antibody arrays is made possible by hybridization-directed binding of specific antibodies to specific photonic crystal elements, as illustrated in FIG. 5. This procedure eliminates the need to robotically deliver different antibody reagents to different array positions. A “universal” set of unique, diverse oligonucleotide sequences is first prepared, and attached to individual photonic crystal elements of a PBG sensor array, to create a “universal PBG-oligonucleotide array” that can be used to prepare any desired antibody array. Each antibody to be placed within the array is conjugated with a specific oligonucleotide that is complementary to only a single oligonuceotide in the “universal PBG-oligonucleotide array.” Oligonucleotides on the PBG and conjugated to the antibodies are selected carefully to be highly specific to their complements, avoiding any possible cross-hybridization with other members of the set. All antibody-oligonucleotide complexes are mixed together and flowed through the PBG sensor array, under specific hybridization conditions, and each antibody is directed by specific hybridization to bind only to sidewall channels of a single photonic crystal element in the sensor array.

In addition to being used to prepare antibody arrays as illustrated in FIG. 5, the “universal PBG-oligonucleotide array” may alternatively be converted to an application-specific oligonucleotide array, by hybridizing with a mixture of “chimeric” DNA probes, each containing at one end a sequence designed to bind to a specific array element, and at the other end, a sequence designed to bind to a specific nucleic acid analyte. The resulting array can then be used for reporter particle-based nucleic acid analysis as described in Example 3, and the array can be recycled for multiple applications, by washing at low ionic strength and elevated temperature to release the bound particles and analyte molecules, followed by hybridization with another mixture of chimeric DNA probes to create a new application-specific array.

In addition to the UV crosslinking method illustrated in FIG. 5, the long DNA strands optionally may be covalently bound to the polyamine-coated surface, or to a variety of reactive silane coatings, using chemical crosslinking employing a variety of crosslinking reagents (Pierce). A further alternative for providing long DNA strands extending between the surface and the analyte-binding entity, i.e., single-stranded DNA tail or antibody, is as follows. The channel sidewalls may be coated with a silane such as aminosilane, then an adapter oligonucleotide pair, bearing a terminal amine group, may be linked to the surface by use of di- or tricarboxylic acid reagent as described previously. Then the restriction fragment and corresponding adapter duplex may be enzymatically ligated. A further modification involves first coating the channel sidewalls with streptavidin, then applying the restriction fragments into which one or a few biotin molecules had been incorporated, via ligation with biotin-containing oligonucleotides or via limited nick translation reaction in the presence of biotinylated nucleotides.

### EXAMPLE 7

### Long Channel Index Contrast Detection and Amplification

For all of the examples described above, it is possible to amplify the signal produced by the target molecules, cells or particles, by increasing the length of the channel allowing additional light to reach the detector. Here, the index contrast provided by the local perturbation of the target must be maintained for the entire length of the channel. The liquid containing the target must also contain, for example, in suspension, non-interacting particles whose index of refraction is different from that of the liquid itself thus producing an effective index of refraction for the mixture n(M). The liquid and particles have index of refraction n(L) and n(P) respectively.

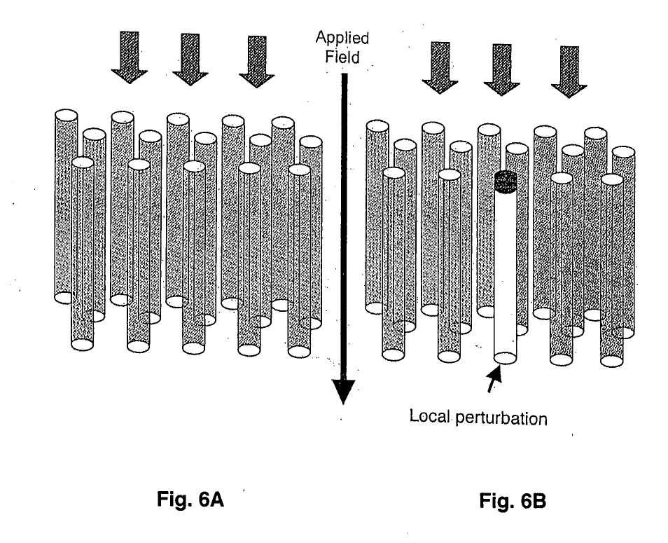

As illustrated in FIGS. 6A-6B, the particles must fit through the channels of the PBG structure and also be small compared to the wavelength of light to minimize Mie scattering. The particles may also exhibit a transport enabling property such as magnetism, charge, weight or other property, providing motion in an applied external magnetic, electric, gravitational or other field. As the liquid containing the particles and targets flows through the PBG channels, FIG. 6A, a band gap is established determined by the index of refraction of the matrix material and effective index of refraction of the liquid mixture, n(M), as shown in FIG. 1B.

When a target is captured, preferably at or near the top of the channel, as in FIG. 6B, the particles continue to flow out of the channel as a result of the applied field leaving behind the liquid filled channel only, with index of refraction n(L). Since the band gap is determined by the index of refraction of the mixture n(M), the channel containing the captured target with index of refraction n(L) breaks the local symmetry of the PBG resulting in detectable light within the spectral region of the band gap as shown in FIG. 1C. The channels in the array can be 100's or 1000's of times longer than the target, hence the cross sectional area of the PBG structure can be increased by that same factor allowing much more light, at the same intensity used in the previous examples, to reach the detector. The total increase in signal, or amplification, is directly proportional to the increase in length I of the channels.

For the examples described above, the size of the targets range from 10's of microns to 10's of nanometers. Therefore, to minimize the effects of Mie scattering by the particles, the particle dimensions should be a factor of 10 to 100 times smaller than the wavelength of light used to probe the targets. A variety of commercially available magnetic, photoconductive, dense; glass, polymer, semiconductor, metal, insulator and other non-interacting nanoparticles currently are available in this size range.

One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A photonic bandgap (PBG) detector, comprising:
one or more photonic crystals having a matrix structure defining a plurality of channels having a length I therethrough;
a fluid within said channel(s) and flowable therethrough;
means for biochemically inducing a detectable increase in light transmission within a bandgap region of the photonic crystal.
means for transmitting light within the bandgap region; and
means for detecting the increase in light transmission therein.

2. The PBG detector of claim 1, further comprising:
an optical filter operably disposed between the photonic crystal and said means for detecting the increase in light transmission.

3. The PBG detector of claim 1, further comprising:
means for applying a variable back pressure to the flow through the channels.

4. The PBG detector of claim 1, wherein the fluid is a gas or a liquid.

5. The PBG detector of claim 4, wherein said gas is moist air, helium, hydrogen, nitrogen, argon, krypton, or sulphurhexafluoride.

6. The PBG detector of claim 4, wherein said liquid is water, a buffer, an ionic solution, a sugar solution or a suspension of nanoparticles having a diameter about 10 to about 100 times less than a wavelength of the transmitted light.

7. The PBG detector of claim 1, wherein said means for biochemically inducing a detectable increase in light transmission comprises a molecule, a cell, or a particle flowably disposed within said channels.

8. The PBG detector of claim 7, wherein the molecule is an inorganic molecule, DNA, RNA, a protein, a toxin, a prion, a peptide, a receptor, or other biomolecule.

9. The PBG detector of claim 7, wherein said cell is a bacterium, archaea, an amoeba, a protist, mycoplasma, a yeast, a microfungus, a microalgae, a microparasite, a lymphocyte, or a subcellular organelle.

10. The PBG detector of claim 7, wherein said particle is a spore or virus.

12. The PBG detector of claim 7, further comprising a first binding agent attached to surfaces of the channels effective to bind said molecule, cell or particle thereto.

13. The PBG detector of claim 12, wherein said first binding agent is DNA, an antibody, a peptide, or a DNA conjugate thereof.

14. The PBG detector of claim 13, wherein said first binding agent is a DNA-antibody conjugate.

15. The PBG detector of claim 14, wherein the DNA comprising said DNA antibody conjugate is hybridized to a DNA attached to the channel surfaces.

16. The PBG detector of claim 12, further comprising a second binding agent attached to a reporter, said second binding agent incapable of binding to the first binding agent and effective to bind said molecule, cell or particle at a site separate from said first binding agent.

17. The PBG detector of claim 16, wherein said reporter is a nanoparticular or microparticular structure.

18. The PBG detector of claim 16, wherein said second binding agent is DNA, an antibody or a peptide.

19. The PBG detector of claim 12, further comprising:
means for enhancing the detectable increase in transmitted light.

20. The PBG detector of claim 19, wherein said means for enhancing the detectable increase in transmitted light comprises:
said plurality of channels having a length greater than said length I;
said fluid including a plurality of flowable nanoparticles having a diameter less than a wavelength of the transmitted light; and
said first binding agent attached to the surfaces of the channels at a position substantially proximate to a top end thereof.

21. The PBG detector of claim 20, wherein said nanoparticles have a diameter about 10 to about 100 times less than a wavelength of the transmitted light.

22. The PBG detector of claim 1, wherein said photonic crystals are arranged in series.

23. A method of optically detecting an analyte, comprising:
flowing an analyte through the channels of the photonic crystal of claim 1;
illuminating the photonic crystal with a light source;
inducing an increase in light transmission within a photonic bandgap of the photonic crystal via binding of one of said analytes to a first binding agent attached to surfaces of the channels and specific for said analyte; and
photodetecting the increase in light transmission within the photonic bandgap thereby optically detecting the analyte.

24. The method of claim 23, wherein said first binding agent is DNA, an antibody, a peptide, or a DNA conjugate thereof.

25. The method of claim 24, wherein said first binding agent is a DNA-antibody conjugate.

26. The method of claim 25, wherein the DNA comprising said DNA antibody conjugate is hybridized to a DNA attached to the channel surfaces.

27. The method of claim 23, wherein said analyte is a bacterium, archaea, an amoeba, a protist, mycoplasma, a spore, a yeast, a microfungus, a microalgae, a microparasite, a virus, a lymphocyte, a prion, a toxin, an inorganic molecule, DNA, RNA, a protein, a peptide, a receptor, or other biomolecule.

28. The method of claim 23, further comprising:
flowing the analyte through the channels of a series of photonic crystals each comprising a different first binding agent effective to bind said one analyte.

29. The method of claim 23, further comprising:
flowing a reporter having a second binding agent effective to bind the analyte attached thereto through the channels, said first and second binding agents effective to bind the analyte concurrently without binding to each other.

30. The method of claim 29, wherein the reporter is a nanoparticular or a microparticular structure.

31. The method of claim 29, wherein said second binding agent is DNA, an antibody or a peptide.

32. The method of claim 23, further comprising:
applying a variable back pressure to the flow through the channels thereby decreasing non-specific analyte binding.

33. A method of identifying the presence of a cell or particle in a biological sample, comprising:
flowing the biological sample through the channels of one or more photonic crystals of claim 1, said channel surfaces comprising one or more antibodies specific for the cell or particle attached thereto;
illuminating said one or more photonic crystals with a light source; and
photodetecting an increase in light transmission within a photonic bandgap of said photonic crystal(s) upon binding of the cell or particle to the one or more antibodies, thereby identifying the cell or particle.

34. The method of claim 33, wherein said antibody is conjugated to a DNA attached to the channel surfaces.

35. The method of claim 34, wherein the DNA comprising said DNA antibody conjugate is hybridized to another DNA attached to the channel surfaces.

36. The method of claim 33, wherein said cell or particle is a bacterium, archaea, an amoeba, a protist, mycoplasma, a spore, a prion, a yeast, a microfungus, a microalgae, a microparasite, a virus, or a lymphocyte.

37. The method of claim 36, wherein said cell or particle is a pathogen.

38. The method of claim 37, wherein said pathogen is E. coli O157:H7 or M. tuberculosis, Bacillus anthracis, Francisella tularensis, Yersinia pestis, Yersinia enterocolitica, Campylobacter jejuni, Listeria monocytogenes, Salmonella species, Shigella species, Vibrio species, Burkholderia mallei, Burkholderia pseudomallei, Coxiella bumetii, Brucella species, Chlamydia species, Coccidioides posadasii, Rickettsia proawzekii, Rickettsia rickettsii, smallpox virus, hemorrhagic fever viruses, encephalitis viruses, yellow fever virus, rabies virus, severe acute respiratory syndrome-associated coronavirus (SARS-CoV), Chikungunya virus, bird flu (H5N1 influenza) virus, Noroviruses, hepatitis viruses, calciviruses, West Nile virus, encephalitis viruseses, prions, Cryptosporidium parvum, Cyclospora cayatanensis, Giardia lambia, Entamoeba histolytica, Toxoplama, and Microsporidia.

39. The method of claim 33, wherein said cell is a lymphocyte from an individual, the method further comprising:
determining the types of antibodies to which the lymphocyte binds to indicate exposure of the individual to a pathogen or to indicate a change in immunocompetent status of the individual.
